(19) United States
(12) Patent Application Publication
(10) Pub. No.: US 2004/0079361 A1
(43) Pub. Date: Apr. 29, 2004

(54) Medicinal aerosols

(76) Inventors: Colin D. Clayton, Leicestershire (GB); John S. Scott, Leicestershire (GB)

Correspondence Address: Ted K Ringsred, Office of Intellectual Property Counsel, 3M Innovative Properties Company, PO Box 33427, St Paul, MN, 55133-3427, US

(21) Appl. No.: 10/467,690

(22) PCT Filed: Jan. 16, 2002
(86) PCT No.: PCT/US02/01502

(30) Foreign Application Priority Data
Jan. 17, 2001 (GB) 0101239.2
Aug. 24, 2001 (GB) 0120658.0

(51) Int. Cl. A61M 11/00

(52) U.S. Cl. 128/200.23

(57) ABSTRACT

A medicinal aerosol product (2) comprising a metal aerosol can (4) equipped with a metering valve (14) and containing a medicinal aerosol formulation, wherein the can is secured within a metal shroud (20) to provide the product with external dimensions substantially equivalent to that of an aerosol can of larger dimensions.

This invention relates to medicinal aerosols and in particular to metered dose inhalers (MDI's) which are used to dispense medicament to the respiratory system of a patient.

Medicinal aerosol formulations in pressurised containers have been available for over forty years. For most of this time, chlorofluorocarbons have been used as the propellants. Drugs have been formulated either as solutions or as suspensions, depending on their solubility properties and other factors. Following environmental concerns over their use, other propellants have been introduced, as this has presented a challenge to reformulate or to introduce new drugs, as well as an opportunity to provide improved pharmaceutical performance.

Two propellants that have emerged as favourites are 1,1,1,2-tetrafluoroethane (HFA 134a) and 1,1,1,2,3,3,3-heptafluoropropane (HFA 227). These have distinctly different solvent properties to the chlorofluorocarbons, and this has had a bearing on the properties of formulations.

When formulating suspensions, micronised drug is dispersed in a propellant system with other ingredients added as appropriate for maintaining the stability of the formulation. One aspect of stability is the homogeneity of the dispersed drug, which can sediment (settle) or cream (float) depending on the density difference between drug and propellant, or it can flocculate, which requires some degree of agitation to deflocculate it. Such challenges are presented when formulating suspensions of any drug, but are particularly important when high potency drugs, such as Formoterol, Fluticasone Propionate, Salmeterol, Procaterol and Ipratropium and salts thereof are formulated.

When more potent drugs are formulated as suspensions, the concentration of drug required is lower than for less potent drugs. Sedimenting, creaming or flocculating drug leads to greater inhomogeneity of the contents that in turn may lead to delivery of incorrect doses when the formulation is dispensed from the metering valve.

In order to ensure optimal through-life dosing of an MDI it is important that the formulation be matched specifically with the aerosol valve and aerosol can. Thus, the concentration of the components of the medicinal aerosol formulation will be selected depending upon the volume of the can from which the formulation will be dispensed and the fill weight i.e. the total number of doses, of the formulation which will be introduced into the can. Rather surprisingly, it has been found that if an identical medicinal aerosol formulation is delivered from an identical metering valve with the same volume of medicinal aerosol formulation contained in cans of different volume there will be a difference in the composition of the dose of formulation delivered from the valve. The degree of this difference depends upon the difference in volume between the two cans and the composition of the medicinal aerosol formulation, particularly the content of the less volatile ingredients. One reason is because if the same volume of an identical aerosol formulation is placed in a larger can there will be a larger unoccupied volume or “head space”. The propellant in the aerosol formulation is the most volatile ingredient and will vaporise to occupy the headspace until there is equilibrium between the liquid and vapour phases. The other ingredients of the aerosol formulation may also vaporise to some extent depending upon the vapor pressure of the ingredient. The result will be that the liquid formulation metered and dispensed by the valve will contain less of the most volatile ingredients and more of the less volatile ingredients compared to the situation when using a can of smaller volume. This concentration of the less volatile ingredients may result in the drug delivery varying from the target dose by an unacceptable margin. Also if the medicinal aerosol formulation comprises relatively non-volatile components, such as ethanol, the concentration of such components may deleteriously affect the spray of droplets which are dispensed. Higher concentrations of ethanol may result in the formation of coarser droplets and the respirable fraction of the dose may be reduced. Correspondingly the particle size distribution determined by Andersen cascade impactor according to methodology in the US Pharmacopoeia, at various stages of emptying of the can, may be affected. The Andersen data may be represented in terms of the throat-to-jet fraction and the fine particle fraction, which includes plates 4 to 6.

MDI's are used in conjunction with an actuator which comprises a housing to contain the aerosol can, a nozzle block which accommodates the valve and directs the dose towards a mouthpiece or nasal adapter through which the patient receives the medicament. The actuator may be of the ‘press-and-breathe’ type which requires that the patient actuates the aerosol manually pressing on the can, or the actuator may be breath-actuated such that the aerosol is fired automatically as the patient inspires through the mouthpiece. It is desirable to standardise the actuator in the interests of patient familiarity and economy of manufacture. However, it is not generally feasible to use the same actuator with aerosol cans of different size even if the cans are equipped with an identical valve. A different size can may affect the airflow within an actuator resulting in different spray characteristics and respirable fraction of the dose delivered. Also, a different size can may not be suitable to interact properly with the triggering mechanism of a breath-actuated actuator.

It is often desirable from a clinical point of view for a doctor to have the option of prescribing MDI's having a different number of total doses e.g. 30, 45, 60, 120, 200 etc. The selection of these may depend upon the nature of the ailment, the dosage regime for the treatment and the expected duration of the treatment etc.

The problems faced with developing medicinal aerosol products having a different number of deliverable doses but delivering the same dose of drug and/or the same fine particle fraction of the dose, is that it is either necessary to develop a range of formulations for use with the same can and valve or it is necessary to use cans of different size and develop actuators for use with each can size.

GB-2267936 discloses an aerosol vial having secured to its outer surface a plastics shroud which covers the base of the vial and extends at least halfway up the sidewall of the vial such that it increases the width of the vial by at least 2 mm and the height of the vial by at least 5 mm. The patent addresses the inherent disadvantages in using small aerosol vials including:

1. insufficient labelling area for including the necessary information

2 handleability

3. compatibility with current adapters, both press-and-breathe and inhalation-actuated.

The patent does not recognise the problem associated with the development of aerosol products having different numbers of total doses to be delivered.

According to one aspect of the present invention there is provided a medicinal aerosol product comprising a metal aerosol can comprising a circular base, cylindrical sidewall extending from the base to an open end to which is attached a metered dose dispensing valve, the aerosol can containing a medicinal aerosol formulation characterised in that the aerosol can is secured within a metal shroud which covers the base of the aerosol can and extends at least partially up the sidewall of the aerosol can to provide the product with external dimensions substantially equivalent to that of an aerosol can of larger dimensions.

According to a second aspect of the present invention there is provided a method of manufacturing a medicinal aerosol product which comprises the steps of:

preparing a medicinal aerosol formulation for use at a first fill weight in a first aerosol can equipped with a metered dose dispensing valve, the first aerosol can comprising a circular base and a cylindrical side wall extending from the base to a shoulder region terminating in a neck to which said valve is attached,

filling said aerosol formulation in a second can which has a volume less than said first can, at a second fill weight which is less than said first fill weight, the second aerosol can comprising a circular base and a cylindrical side extending from the base to a shoulder region terminating in a neck to which said metered dose dispensing valve is attached, the aerosol formulation being filled at a second fill weight which is less than said first fill weight such that when dispensed from said second can through said metered-dose dispensing valve the emitted dose will exhibit pharmaceutical characteristics substantially identical to the dose characteristics obtained if said formulation had been filled at said first fill weight in said first can and dispensed therefrom through said metered dose dispensing valve,

wherein said second can is secured within a metal shroud which covers the base of the can and extends at least partially up the sidewall to provide the second with external dimensions substantially identical to said first can.

The invention further provides a method of producing a second metered dose inhaler product comprising a medicinal aerosol formulation, aerosol container, valve and actuator that produces fewer doses than a first metered dose inhaler product, where the dose delivery characteristics, formulation, valve size and actuator are the same in the second product as the first product, by using less of the formulation in a smaller aerosol container for the second product, in conjunction with an adaptor to permit the smaller aerosol container to fit properly in the actuator. The adapter is generally constructed and arranged so that the combination of the adapter and smaller aerosol container has substantially the same outer dimension as that of the first metered dose inhaler product.

The pharmaceutical characteristics defined here are the amounts of drug delivered per dose and/or the throat-to-jet and fine particle fractions of Andersen data at various stages of emptying of the can.

The invention provides a simple and effective system which enables an identical medicinal aerosol formulation to be used in a range of aerosol products having different fill weights, which products may be used in the same actuator. Furthermore, the products may be manufactured using the same automatic machinery and the same size labelling and/or packaging may also be used in conjunction with the product.

When producing medicinal aerosol formulations dispensing the same dose of medicament but having different fill weights i.e. total number of doses, it is desirable to use an identical metering valve. As a result the dimensions of the neck portion of the aerosol can must be the same and therefore the major difference in the external dimensions of aerosol cans of different volume is the length of the sidewall. It has been found that the body of a larger size aerosol can can be configured such that it may act as shroud for a smaller volume aerosol can to result in an aerosol product which essentially mimics the outer dimensions of an aerosol product made from the larger can. The use of the body of a larger can to form such a shroud has significant advantages since the resulting shrouded can will have dimensions and properties which are not significantly different from a product formed from the larger can. Thus, the shrouded can may readily be handled on the automatic lines designed to handle the larger can since the weight, dimensions and frictional properties of the shrouded can are similar. Similarly, the outer surface of the shrouded can will allow use of the same size labels as the larger can and the external dimensions of the aerosol product formed with the shrouded can will allow use of the same packaging and packaging machinery. The aerosol product formed of the shrouded can may be used in the actuators designed for use with the larger can with comparable performance. Thus, the airflow characteristics will not alter substantially since the shrouded can has substantially identical dimensions to the product formed of the larger can and the shrouded can will fit within a breath actuated actuator and co-operate with the triggering mechanism in the same manner as an aerosol product formed from the larger can.

While it is readily feasible to manufacture special shrouds to mimic the size of a larger can, in practice, since the larger cans are readily available, it is convenient to simply remove the top portion of the can from a region just below the shoulder. This can readily be achieved with a conventional parting-off tool e.g. on a standard centre lathe. The sharp edges at the open end of the can may be removed with a de-burring tool, abrasive paper etc.

In order to properly locate the can within the shroud formed from the larger can, it is convenient to form a circumferential bead which acts as a stop once the smaller can is pushed inside the shroud. A bead may be conveniently formed by a roller urged against the outside of the larger can to form a circumferential recess in the outside surface of the can resulting in a bead on the inner surface of the can. Again, this operation may be conducted on a standard centre lathe e.g. utilising a roller and support peg so that the bead on the can can be consistently reproduced. The roller and peg are mounted on the centre lathe so that the bead can be formed with the required degree of accuracy. The can is simply placed on to the rotating support peg, after which the roller is moved into contact with the outside wall of the can and then pushed in slightly to form a bead.

The inner can is then placed within the shroud so that the base of the inner can contacts the bead. The shroud may be secured to the inner can by deformation of the shroud to obtain an interference fit. In one embodiment the interference fit is achieved by means of three or more dimples pushed in to the outside of the shroud such that the internal surface of the shroud in the region of the dimples is forced against the external surface of the can. The dimples may readily be formed by a punch and die arrangement, the depth of the dimples being controlled using a stop mechanism. Preferably, the dimples are arranged symmetrically around the circumference of the shroud such that if three dimples are used they will be spaced approximately 120° apart.

In accordance with a preferred embodiment the interference fit is obtained by reducing the diameter of the shroud e.g. by use of a recone punch and die. The recone punch and die may conveniently be fitted to a Fly-Press so that the shrouds may be aligned and drawn down to a set length each time. This technique secures the shroud firmly to the can with a water-tight seal such that the shrouded can may be immersed in a water bath without ingress of water between the can and the shroud.

The shroud must extend up the sidewall of the can for a sufficient length to allow formation of an interference fit. Preferably, the shroud extends substantially to the shoulder region of the can to provide a substantially continuous outer sidewall to the aerosol product.

If necessary, the base of the shroud may comprise an aperture to ensure that any air trapped between the can and the shroud is at ambient pressure.

In principle, the can size is chosen according to the required fill weight. A range of can sizes can be filled with aerosol formulation from a batch, then tested for dose characteristics. The can size with the closest match to the comparative product is then selected.

It is useful to specify can sizes by the volume of water that they contain after a valve has been placed on and removed from a brimful can. Typical values (in millilitres) are 8.0, 10.3, 13.6, 14.2 and 16.25. In a preferred example of the invention, a comparative product of 120 doses in a 16.25 ml can has matched dose characteristics with a product of 60 doses in an 8.0 ml can, the 8.0 ml can being enshrouded with a shroud made from a 16.25 ml can.

The selection of the aerosol formulation, fill weight and volume of the can can readily be determined by a person skilled in the art. The invention finds particular utility for use with formulations comprising non-volatile ingredients, such as ethanol, which have been found to be more problematic than formulations which simply consist of aerosol propellant and drug.

The invention will now be described with reference to the accompanying drawings in which:

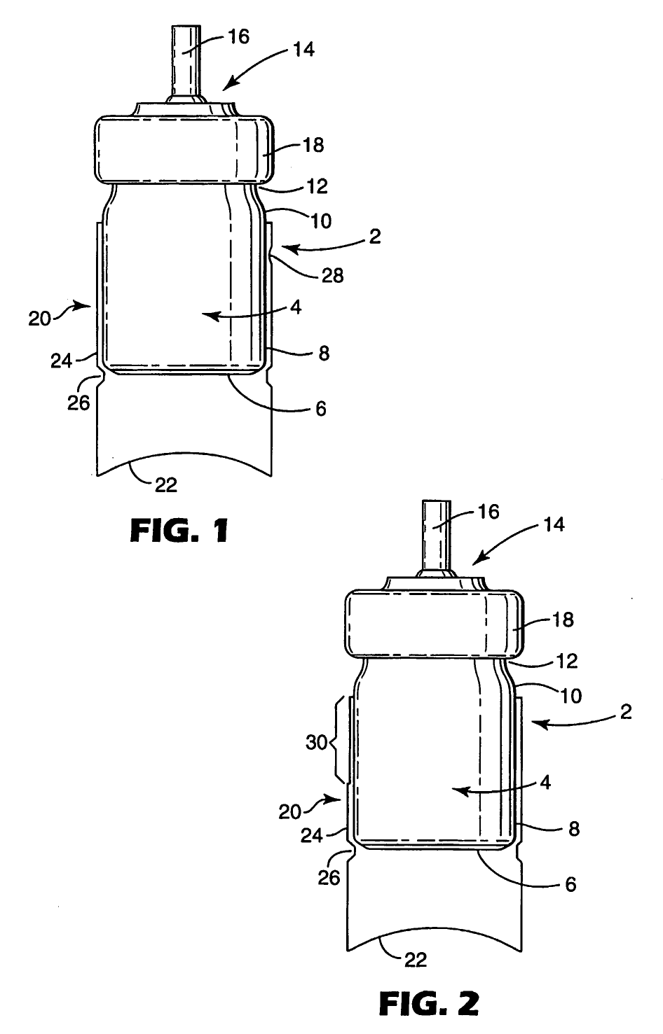

FIG. 1 represents a diagram of an aerosol product in accordance with the invention,

FIG. 2 represents a diagram of a further aerosol product in accordance with the invention and

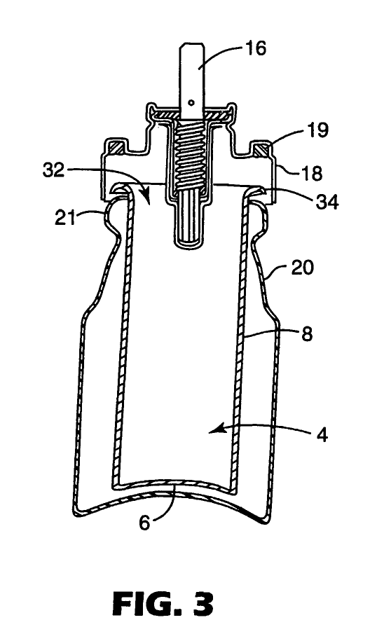

FIG. 3 represents a diagram of a further aerosol product in accordance with the invention.

In the figures, like numerals represent like parts.

The aerosol product generally shown at 2 comprises an aerosol can 4 having a circular base 6 and a cylindrical sidewall 8 extending from the base 6 to a shoulder region 10 terminating in a neck 12. A metered dose dispensing valve generally shown at 14 comprises a valve stem 16 and a valve ferrule 18 which is crimped to the neck 12 of the aerosol can. The aerosol can is generally made from aluminium e.g. by drawing.

The aerosol can 4 is secured within a shroud generally shown at 20. The shroud is formed from an aerosol can of larger size and comprises a circular base 22 and cylindrical sidewall 24. A circumferential bead 26 is formed in the sidewall 24 to act as a stop for the aerosol can 4. The base 6 of the aerosol can 4 abuts the bead 6 and prevents the can 4 from further movement into the shroud 20. The cylindrical sidewall 24 of the shroud extends to the shoulder region 10 of the aerosol can 4.

The aerosol can 4 is secured to the shroud 20 by means of three dimples 28 pressed into the shroud such that the internal surface of the shroud is forced into contact with the external surface of the aerosol can 4 in the region of the dimples 28.

In practice the aerosol can and shroud are secured together prior to aerosol formulation being introduced into the aerosol can and fitting of the metered dose dispensing valve 14. Thus, the shrouded can may be used on a handling line and with all of the automatic handling machinery designed for use with an aerosol can from which the shroud 20 is made.

The embodiment shown in FIG. 2 differs from that shown in FIG. 1 in that the shroud 20 is secured to the aerosol can 4 by reducing the diameter of the sidewall 20 of the shroud in the region 30 such that it forms an interference fit with the aerosol can 4. The diameter reduction may be achieved by means of a recone punch and die. The shrouded can may then be used in the identical manner to that described with reference to FIG. 1.

An advantage of the embodiments of the invention over that of plastic shrouds of the prior art is that a greater force is needed to separate the shroud from the can, even further limiting the possibility of them becoming separated during or after manufacture. A particular advantage of the embodiment shown in FIG. 2 is that it is possible to make a water-tight seal between the shroud and the can in the region 30, so that water does not enter the space between the shroud and can during waterbath testing of the filled can.

FIG. 3 represents an exploded view of a further embodiment in accordance with the invention. In this embodiment the shroud 20 is in the form of an aerosol can of larger size to the can 4 in which the aerosol formulation is contained. The can 4 comprises the circular base 6 and a cylindrical sidewall 8 extending from the base to an open end 32. The open end 32 comprises a circumferential flange 34 which is dimensioned to extend over the neck 21 of the larger aerosol can which is the shroud 20. The valve ferrule 18 is crimped over the neck 21 trapping the circumferential flange 34 between the ferrule and the neck thereby firmly securing the aerosol can 4 within the shroud 20. The gasket seal 19 of the aerosol valve forms a seal with the circumferential flange 34.

This embodiment has the advantage that it is compatible with the existing assembly and filling equipment and that the post-manufacture handling and uses e.g. function testing, labelling etc. remain unchanged. Furthermore, the design is tamper proof and it is not apparent to the patient that there is a smaller container within the shroud.

1. A medicinal aerosol product comprising a metal aerosol can comprising a circular base, cylindrical sidewall extending from the base to an open end to which is attached a metered dose dispensing valve, the aerosol can containing a medicinal aerosol formulation characterised in that the aerosol can is secured within a metal shroud which covers the base of the aerosol can and extends at least partially up the sidewall of the aerosol can to provide the product with external dimensions substantially equivalent to that of an aerosol can of larger dimensions.

2. A medicinal aerosol product as claimed in claim 1 in which the metal aerosol can comprises a circular base, cylindrical sidewall extending from the base to an open end shoulder region and a neck.

3. A medicinal aerosol product as claimed in claim 1 or claim 2 in which the shroud is derived from said aerosol can of larger dimensions.

4. A medicinal aerosol product as claimed in any preceding claim in which the aerosol can is an interference fit within the shroud.

5. A medicinal aerosol product as claimed in claim 4 in which the shroud comprises a cylindrical sidewall having at least three dimples forming said interference fit with the external surface of the sidewall of the aerosol can.

6. A medicinal aerosol product as claimed in claim 4 in which the shroud comprises a cylindrical sidewall having an internal diameter which is an interference fit with the external surface of the sidewall of the aerosol can.

7. A medicinal aerosol product as claimed in any preceding claim in which the shroud comprises a sidewall having a circumferential bead acting as a stop to the base of the aerosol can.

8. A medicinal product as claimed in claim 1 in which the shroud is an aerosol can of larger dimensions comprising a circular base, cylindrical sidewall extending from the base to a shoulder region and a neck; the aerosol can comprises a circumferential lip extending around its open end and over the neck of the shroud and the metered dose dispensing valve is secured to the neck of the shroud and forms a seal with the circumferential lip of the aerosol can.

9. A medicinal aerosol product as claimed in any preceding claim in which the aerosol can has a volume of about 5 ml and the external dimensions of the product is equivalent to that of an aerosol can having a volume of about 10 ml or about 15 ml.

10. A medicinal aerosol product as claimed in any preceding claim in which the aerosol formulation comprises propellant 134a and/or propellant 227.

11. A medicinal aerosol product as claimed in any preceding claim in which the aerosol formulation comprises ethanol.

12. A medicinal aerosol product as claimed in claim 1 substantially as herein described with reference to the accompanying drawings.

13. A method of manufacturing a medicinal aerosol product which comprises the steps of:
preparing a medicinal aerosol formulation for use at a first fill weight in a first aerosol can equipped with a metered dose dispensing valve, the first aerosol can comprising a circular base and a cylindrical side wall extending from the base to an open end to which said valve is attached,
filling said aerosol formulation in a second can which has a volume less than said first can, at a second fill weight which is less than said first fill weight, the second aerosol can comprising a circular base and a cylindrical side extending from the base to an open end to which said metered dose dispensing valve is attached, the aerosol formulation being filled at a second fill weight which is less than said first fill weight such that when dispensed from said second can through said metered-dose dispensing valve the emitted dose will exhibit pharmaceutical characteristics substantially identical to the dose characteristics obtained if said formulation had been filled at said first fill weight in said first can and dispensed the refrom through said metered dose dispensing valve,
wherein said second can is secured within a metal shroud which covers the base of the can and extends at least partially up the sidewall to provide the second with external dimensions substantially identical to said first can.

14. A method of manufacturing a medicinal method aerosol product as claimed in claim 13 in which the aerosol can is an interference fit within the shroud.

15. A method of manufacturing a medicinal method aerosol product as claimed in claim 14 in which the shroud comprises a cylindrical sidewall having at least three dimples forming said interference fit with the external surface of the sidewall of the aerosol can.

16. A method of manufacturing a medicinal method aerosol product as claimed in claim 14 in which the shroud comprises a cylindrical sidewall having an internal diameter which is an interference fit with the external surface of the sidewall of the aerosol can.

17. A method of manufacturing a medicinal method aerosol product as claimed in any one of claims 13 to 16 in which the shroud comprises a sidewall having a circumferential bead acting as a stop to the base of the aerosol container.

18. A method of manufacturing a medicinal method aerosol product as claimed in claim 13 in which the shroud is an aerosol can of larger dimensions comprising a circular base, cylindrical sidewall extending from the base to a shoulder region and a neck; the aerosol can comprises a circumferential lip extending around its open end and over the neck of the shroud and the metered dose dispensing valve is secured to the neck of the shroud and forms a seal with the circumferential lip of the aerosol can.

19. A method of manufacturing a medicinal method aerosol product as claimed in claims 13 to 18 in which said first aerosol can has a volume of about 10 ml or about 15 ml and said second aerosol can has a volume of about 5 ml.

20. A method of manufacturing a medicinal aerosol product as claimed in any one of claims 13 to 19 in which the aerosol formulation comprises propellant 134a and/or propellant 227.

21. A method of manufacturing a medicinal aerosol product as claimed in any one of claims 13 to 20 in which the aerosol formulation comprises ethanol.

22. A method of manufacturing a medicinal aerosol product substantially as herein described with reference to the accompanying drawings.

23. A method of producing a second metered dose inhaler product comprising a medicinal aerosol formulation, aerosol container, valve and actuator that produces fewer doses than a first metered dose inhaler product, where the dose delivery characteristics, formulation, valve size and actuator are the same in the second product as the first product, by using less of the formulation in a smaller aerosol container for the second product, in conjunction with an adapter to permit the smaller aerosol container to fit properly in the actuator.
